(19) United States
(12) Patent Application Publication
(10) Pub. No.: US 2006/0020252 A1
(43) Pub. Date: Jan. 26, 2006

(54) Self-contained disposal assembly

(76) Inventor: Michael David Strong, Taylorsville, UT (US)

Correspondence Address: RADER, FISHMAN & GARAUER PLLC, 10653 SOUTH RIVER FRONT PARKWAY, SUITE 150, SOUTH JORDAN, UT, 84095, US

(21) Appl. No.: 10/895,516

(22) Filed: Jul. 20, 2004

(51) Int. Cl. A61F 13/15

(52) U.S. Cl. 604/385.13

(57) ABSTRACT

A self-contained disposal assembly includes a containment compartment configured to be attached to a diaper; and an attached disposal bag fixedly coupled to the diaper wherein the attached disposable bag is configured to be disposed within the containment compartment and is further configured to envelop the diaper.

### TECHNICAL FIELD

The present disclosure relates to disposable diapers and more specifically to accessories included with diapers.

### BACKGROUND

The use and function of disposable diaper pants is well known. Conventional disposable diapers frequently include an absorbent pad, an impermeable backing and a fastening system. When the diaper is soiled, the diaper is removed from the person and thrown away. If the diaper is simply left after it has been changed, the diaper can emit foul odors, either from odors from the initial contents of the soiled diaper or from odors caused as the diaper sits. Consequently, in order to minimize foul odors the diaper is often either removed from the room to an outdoor container or treated in some other manner.

One such manner of treating a soiled diaper includes placing the soiled diaper in a plastic sack to individually seal each diaper. According to such an approach, the diaper is placed into a large plastic bag. The bag is then twisted to seal and isolate the diaper and thus isolate the odor. Such systems are often of such a size that the systems are not carried with the user. As a result, when a parent or other care taker is away from home, they are unable to find an acceptable way to dispose of a diaper such that the diaper will not emit foul odors.

Another approach is to place the diaper in a separate sealable container, such as a sealable plastic bag. This often requires that the parent or caretaker leave the child to find the plastic bag. Further, even in the event that a sealable container is available, it is often inconvenient to find the sealable receptacle while the child is in the process of being changed. This is often inconvenient and troublesome as the child may thus be left in a vulnerable position and interrupt the diaper changing process.

Disposal of the soiled diaper may also be inconvenient. For example, in some circumstances time may not permit a parent or caretaker to remove the soiled diaper to an outdoor disposal location. Further, in some locations it may not be convenient or even possible to find a sealable container for sealing the soiled diaper to minimize odor.

Several approaches have been attempted to provide such a system. For example, some approaches have made use of a plastic bag that is provided with the diaper. In the past, these separate removable plastic disposal bags have been included in pouches on the outside of the diaper. Accordingly, when the diaper is soiled, the separate disposal bag is removed from the pouch and the soiled diaper is placed therein. In addition to being unsightly, the separate disposal bags can be difficult to use as they may be hard to open and control with one hand. Further, the separate disposal bags may become separated from the diaper and lost, thereby minimizing the benefit of including a disposal bag.

### SUMMARY

A self-contained disposal assembly includes a containment compartment configured to be attached to a diaper and an attached disposal bag fixedly coupled to the diaper wherein the attached disposable bag is configured to be disposed within the containment compartment and is further configured to envelop the diaper. The use of an attached disposal bag that is contained within a containment compartment allows for the discrete inclusion of a disposal assembly that reduces the mess and odor often associated with the disposal of diapers.

### BRIEF DESCRIPTION OF THE DRAWINGS

The accompanying drawings illustrate various embodiments of the present invention and are a part of the specification. The illustrated embodiments are merely examples of the present assemblies and do not limit the scope of the disclosure.

FIG. 1 is a rear view of a diaper having a self-contained disposal assembly in which an attached disposal bag is shown contained within a containment compartment that is located between the impermeable cover and the absorbent pad of the diaper.

FIG. 2 is a rear view of the diaper shown in FIG. 1 in which the attached disposal bag is shown removed from the containment compartment.

FIG. 3 is a rear view of the diaper shown in FIGS. 1-2 in which the attached disposal bag is shown enveloping the diaper, which has been rolled up for disposal.

FIG. 4 is a frontal view of the rolled up diaper shown in FIG. 3 in which the attached disposal bag is sealed.

FIG. 5 is the rear view of a diaper having a self-contained disposal assembly in which an attached disposal bag is shown coupled near the waste band of the diaper.

FIG. 6 is a rear view of a diaper having a self-contained disposal assembly in which an attached disposal bag is shown contained within a containment compartment attached to the outside of the impermeable cover of the diaper.

Throughout the drawings, identical reference numbers designate similar, but not necessarily identical, elements.

### DETAILED DESCRIPTION

A diaper is provided that includes a self-contained disposal assembly. The self-contained disposal assembly includes a disposal bag that is configured to be placed within a containment compartment until the attached disposal bag is needed. The use of an attached disposal bag that is contained within a containment compartment allows for the discrete inclusion of a disposal assembly that reduces the mess and odor often associated with the disposal of diapers.

In the following description, for purposes of explanation, numerous specific details are set forth in order to provide a thorough understanding of the present assembly. It will be apparent, however, to one skilled in the art that the present method and apparatus may be practiced without these specific details. Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment.

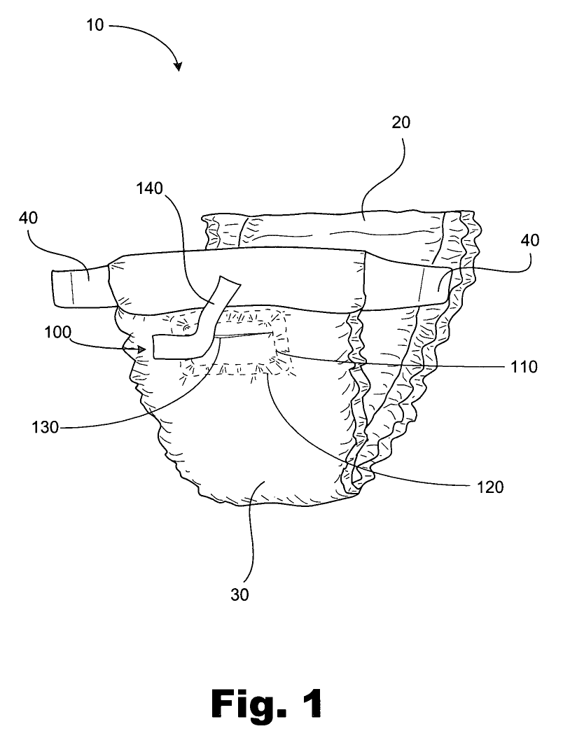

FIG. 1 is a rear view of a diaper (10) having a self-contained disposal assembly (100) located in the rear of the diaper (10). The diaper generally includes an absorbent pad (20) attached to an impermeable cover (30), as is well known in the art. Tabs (40) are coupled to the rear of the diaper (10) for securing the diaper (10) to a person. As will be discussed in more detail below, the self-contained disposal assembly (100) provides for convenient disposal of the diaper (10) while minimizing the odor and/or mess frequently associated with the disposal of diapers.

The self-contained disposal assembly (100) includes an attached disposal bag (110) located within a containment compartment (120). The containment compartment (120) shown is located between the absorbent pad (20) and the impermeable cover (30), such that the self-contained disposal assembly (100) is substantially concealed by the impermeable cover.

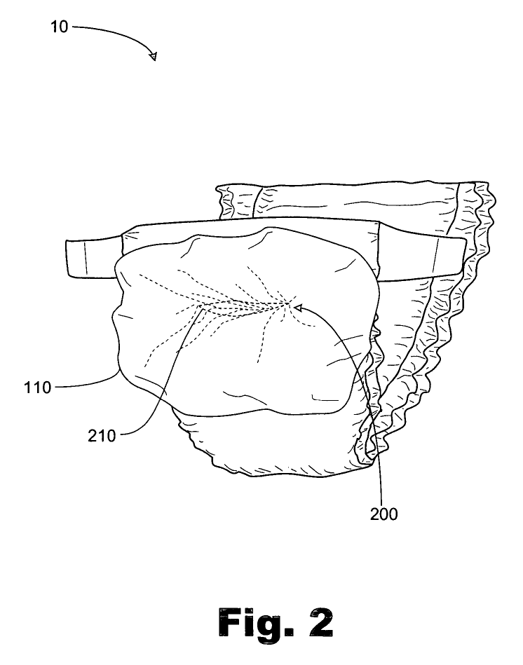

The containment compartment (120) is accessible through an opening (130) defined in the rear portion of the impermeable cover (30). The attached disposal bag (110) is diverse from the containment compartment (120), is configured to be contained therein, and may be attached thereto. As shown in FIGS. 2-5, the attached disposal bag (110) is directly attached to the containment compartment (120).

In other embodiments, the attached disposal bag (110) may otherwise be attached to the diaper (10), provided the attached disposal bag (110) is fixedly attached at some point to the diaper. Accordingly, the attached disposal bag (110) may be attached to the containment compartment (120) or to some other location on the diaper (10). Further, the attached disposal bag (110) may be scented or otherwise treated.

The attached disposal bag (110) may be thus attached by any suitable methods, including by the use of adhesives, by ultrasonic welding, or any other suitable methods. The containment compartment (120) may be formed of an impermeable material, such that liquids that seep through the absorbent pad are not able to escape through the opening (130). The attached disposal bag (110) may be formed of any suitable material, such as thin, flexible plastic materials or any other suitable materials.

As introduced, the attached disposal bag (110) contained within the containment compartment (120) is accessible through the opening (130). As seen in FIG. 1, the attached disposal bag (110) is folded down to a reduced size in order to fit closely within the containment compartment (120). While the attached disposal bag (110) is thus folded and contained within the containment compartment (120), the entire self-contained disposal assembly (100) is well concealed. The opening (130) may be further concealed by a cover, such as the adhesive tab (140).

As shown in FIG. 1, the adhesive tab (140) is placed over the opening (130). In addition to providing additional concealment for the self-contained disposal assembly (100), the use of a cover, such as the adhesive tab (140), helps ensure that the attached disposal bag (110) remains within the containment compartment (120) until the attached disposal bag (110) is to be used. While the use of an adhesive tab (140) has been described to cover the opening (130) and/or contain the attached disposal bag (110), those of skill in the art will appreciate that other covers may be used or provided to enhance the concealment of the assembly (100) and/or the containment of the disposal bag (110).

Accordingly, as shown in FIG. 1, the self-contained disposal assembly (100) discretely contains the attached disposal bag (110) within the containment compartment (120) while providing convenient access to the attached disposal bag (110). The attached disposal bag (110) in turn provides for the convenient sealing of a soiled diaper, thereby reducing the need to find a separate external bag. Further, the attachment of the attached disposal bag (110) reduces the possibility that the attached disposal bag (110) will become separated from the diaper (10).

In addition, the configuration of the attached disposal bag (110) eases the placement of the diaper (10) therein. The placement of the diaper (10) within the attached disposal bag (110) may thus be eased, at least in part, due to the relative size of the attached disposal bag (110). For example, as will be discussed in more detail below, the attached disposal bag (110) is configured to readily expand to envelop the diaper (10).

FIG. 2 shows the attached disposal bag (110) as it is removed from the containment compartment (120; FIG. 1). The attached disposal bag (110) is expandable from its collapsed size. The attached disposal bag (110) includes a bag opening (200) defined therein. The exemplary attached disposal bag (110) shown includes a cinch closure (210) coupled to the bag opening (200).

The attached disposal bag (110) is easily removed from the containment compartment (120; FIG. 1) by removing the adhesive tab (140; FIG. 1) and grasping the attached disposal bag (110) through the opening (130; FIG. 1). The fixed attachment of the disposal bag (110) to the diaper (10) minimizes the possibility that the attached disposal bag (110) will become separated from the diaper (10).

In particular, as the attached disposal bag (110) is withdrawn from its location inside the containment compartment (120; FIG. 1), the attachment of the attached disposal bag (110) to the diaper (10) and/or the containment compartment (120) prevents the attached disposal bag (110) from being separated from the diaper (10). As a result, the attachment of the attached disposal bag (110) keeps the attached disposal bag (110) close at hand. As will be discussed in more detail, once the attached disposal bag (110) is removed from within the containment compartment (120; FIG. 1), the attached disposal bag may then be used to surround and seal the soiled diaper.

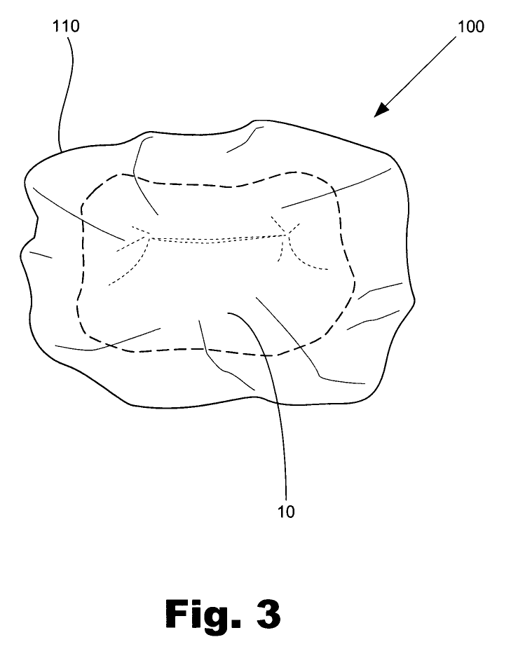

FIG. 3 is a rear view of a diaper (100) having a self-contained disposal assembly (100) in which the diaper (10) is folded and placed within the attached disposal bag (110). Frequently, when a diaper (10) is soiled, the diaper (10) is collapsed by rolling the diaper, beginning with the front of the diaper (10). The diaper (10) is then maintained thus rolled by securing the tabs (40; FIG. 1) to the impermeable cover (30; FIG. 1).

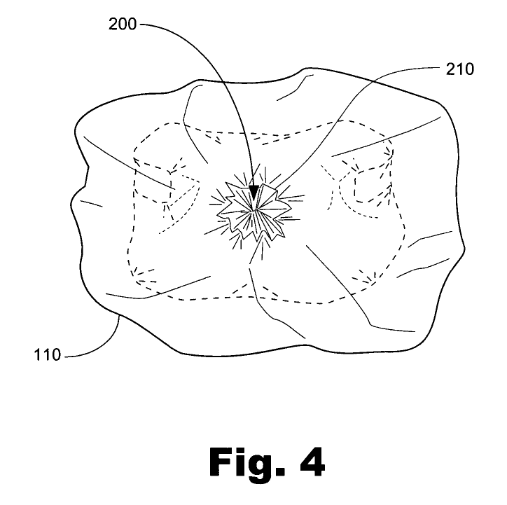

As shown in FIG. 3, the diaper (10) is rolled up and enveloped by the attached disposal bag (110). To envelop the rolled up diaper (10) with the attached disposal bag (110), the attached disposal bag (110) is first withdrawn from the containment compartment (120; FIG. 2) as previously discussed. Then the attached disposal bag (110) is turned inside out while maintaining the diaper (10) inside the attached disposal bag (110). As a result, the rear of the diaper (10) becomes the bottom of the inside-out attached disposal bag (110). With the diaper (10) thus placed within the attached disposal bag (110), the self-contained disposal assembly (100) is ready to be sealed around the diaper, as will now be discussed with reference to FIG. 4.

FIG. 4 is a frontal view of the diaper (10) enveloped by the attached disposal bag (110). To seal the exemplary attached disposal bag (110) the cinch closure (210) is pulled tight to close the bag opening (200). Accordingly, the diaper (10) may thus be easily placed and sealed within the convenient attached disposal bag (110). Once the diaper (10) is thus sealed away, the diaper may then be disposed of in a convenient manner while minimizing odors. As a result, the diaper (10) may then be placed in indoor garbage receptacles until such time as the garbage receptacle is emptied.

While the attached disposal bag shown in FIGS. 1-4 is closed with a cinch closure (210), other types of closures may also be used. Such closures may include, but are in no way limited to, re-sealable interlocking closures such as those frequently used in sandwich bags, hook and loop type closures, and/or adhesive tabs. Further, those of skill in the art will appreciate that the containment compartment may be configured in many different ways and/or located in many different locations.

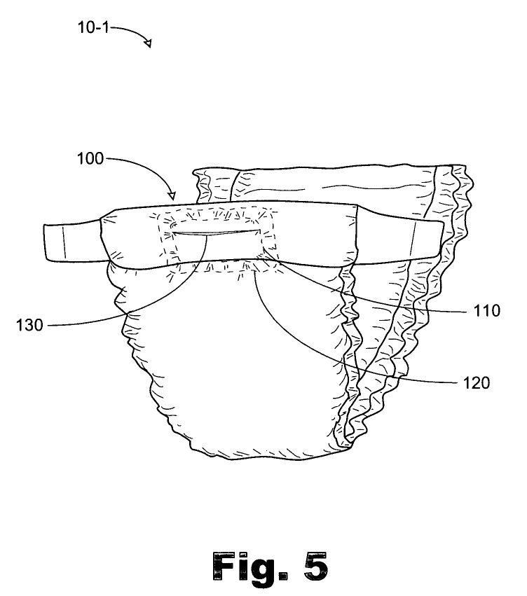

For example, as shown in FIG. 5, a diaper (10-1) may have a self-contained disposal assembly (100) located near the top and rear thereof. This exemplary diaper (10-1) illustrates that the self-contained disposal assembly (100) may be located almost anywhere on the diaper (10-1).

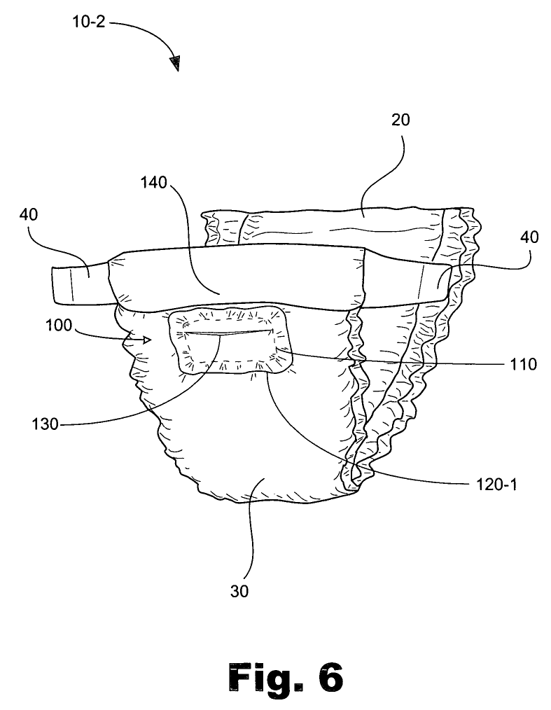

Further, as shown in FIG. 6, the containment compartment (120-1) may be placed on the outside of the impermeable cover (30). In such a configuration, an opening (130-1) is formed on the outside of the containment compartment (120-1). In such an implementation, the attached disposal bag (110) is placed within the containment compartment (120-1) and is fixedly coupled at some point or points to either the containment compartment (120-1) or the diaper (10-2) at other locations, in a similar manner as discussed above with reference to FIG. 1.

In conclusion, a diaper is provided that includes a self-contained disposal assembly. The self-contained disposal assembly includes a disposal bag that is configured to be placed within a containment compartment until the attached disposal bag is needed. The use of an attached disposal bag that is contained within a containment compartment allows for the discrete inclusion of a disposal assembly that reduces the mess and odor often associated with the disposal of diapers.

The preceding description has been presented only to illustrate and describe embodiments of the present assemblies and diapers. It is not intended to be exhaustive or to limit the disclosure to any precise form disclosed. Many modifications and variations are possible in light of the above teaching. It is intended that the scope of the invention be defined by the following claims.

What is claimed is:

1. A self-contained disposal assembly, comprising:
a containment compartment configured to be attached to a diaper; and
an attached disposal bag fixedly coupled to said diaper wherein said attached disposable bag is configured to be disposed within said containment compartment and is further configured to envelop said diaper.

2. The assembly of claim 1, wherein said attached disposal bag is fixedly connected to said containment compartment.

3. The assembly of claim 1, wherein said attached disposal bag has an opening defined therein and a closure coupled to said opening.

4. A diaper, comprising:
an absorbent pad;
a cover coupled to said absorbent pad;
a containment compartment coupled to said cover; and
an attached disposal bag fixedly coupled to said cover and wherein said attached disposal bag is configured to be placed within said containment compartment and is further configured to at least partially envelop said absorbent pad and said cover.

5. The diaper of claim 4, and wherein said containment compartment is located between said absorbent pad and said cover.

6. The diaper of claim 5, and wherein said cover includes an opening defined therein for providing access to said attached disposal bag when said attached disposal bag is located within said containment compartment.

7. The diaper of claim 6, and further comprising an opening cover placed over said opening.

8. The diaper of claim 7, and wherein said opening cover comprises an adhesive tab.

9. The diaper of claim 4, and wherein said containment compartment is located on an outside portion of said cover.

10. The diaper of claim 4, and wherein said attached disposal bag has a bag opening defined therein and a closure coupled to said opening.

11. The diaper of claim 10, and wherein said closure comprises a cinch type closure.

12. The diaper of claim 10, and wherein said closure comprises a resealable interlocking channel type closure.

13. The diaper of claim 4, and wherein said attached disposal bag is fixedly attached to said containment compartment.

14. The diaper of claim 4, and wherein said attached disposal bag is fixedly attached to said cover.

15. A diaper, comprising:
an absorbent pad;
a cover coupled to said absorbent pad;
a containment compartment disposed between said cover and said absorbent pad;
an attached disposal bag fixedly coupled to said cover and wherein said attached disposal bag is configured to be placed within said containment compartment and is further configured to at least partially envelop said absorbent pad and said cover; and
an opening defined in said cover for providing access to said containment compartment.

16. The diaper of claim 15, and further comprising an opening cover covering said opening.

17. The diaper of claim 16, and wherein said opening cover includes an adhesive tab.

18. The diaper of claim 15, and wherein said containment compartment is disposed in a rear portion of said diaper.

19. The diaper of claim 15, and wherein said attached disposal bag is scented.
